(19) United States
(12) Patent Application Publication
(10) Pub. No.: US 2007/0270702 A1
(43) Pub. Date: Nov. 22, 2007

(54) Portable electronic device

(75) Inventor: Onni Ahola, Oulu (FI)

Correspondence Address: HOFFMANN & BARON, LLP, 6900 JERICHO TURNPIKE, SYOSSET, NY, 11791, US

(73) Assignee: Polar Electro Oy

(21) Appl. No.: 11/803,841

(22) Filed: May 16, 2007

(30) Foreign Application Priority Data
May 18, 2006 (FI) 20065335

(51) Int. Cl. A61B 5/02

(52) U.S. Cl. 600/502

(57) ABSTRACT

The invention relates to a portable electronic device comprising: an aperture for allowing surrounding optical radiation modulated by a user's tissue to access the portable electronic device; a radiation detector for generating an electrical signal from the surrounding optical radiation modulated by the user's tissue; and a signal processing chain connected to the radiation detector and configured to generate blood pressure pulse information from the electrical signal.

### CROSS-REFERENCE TO RELATED APPLICATION

This application claims priority to Finnish Patent Application Serial No. 20065335, filed on May 18, 2006, which is incorporated herein by reference.

### BACKGROUND OF THE INVENTION

1. Field of the Invention

The invention relates to a portable electronic device for optically measuring a blood pressure pulse from a user's tissue.

2. Description of the Related Art

In optical measurement of a blood pressure pulse, a subject's tissue is lit by a radiation source of a measuring system, and radiation scattering from the tissue is detected by a radiation detector. Scattering radiation involves a modulated component whose modulation depends on a blood pressure pulse occurring in the tissue.

Drawbacks of prior art solutions include complexity of the measurement arrangement, which is caused by the radiation source, and power consumption, which make the measurement of the blood pressure pulse difficult to apply to a portable electronic device. It is thus advantageous to examine other ways of implementing the measurement of a blood pressure pulse in a portable electronic device.

### SUMMARY OF THE INVENTION

An object of the invention is to provide a portable electronic device so as to achieve a simple measurement of a blood pressure pulse with a low power consumption. This is achieved by a portable electronic device comprising an aperture for allowing surrounding optical radiation modulated by the user's tissue to access the portable electronic device; a radiation detector for generating an electrical signal from the surrounding optical radiation modulated by the user's tissue; and a signal processing chain connected to the radiation detector and configured to generate blood pressure pulse information from the electrical signal.

Preferred embodiments of the invention are disclosed in the dependent claims.

The idea underlying the invention is that the measurement of a blood pressure pulse utilizes optical radiation which originates from the surroundings and which is modulated as a result of a blood pressure pulse occurring in a user's tissue. The use of surrounding radiation is made possible by a suitable configuration of an aperture, radiation detector and a signal processing chain.

Several advantages are achieved by the portable electronic device according to the invention. An advantage achieved is measurement of a blood pressure pulse which does not necessitate a radiation source to be provided in the portable electronic device. Consequently, the structure of the measuring arrangement becomes simpler, and generation of optical radiation to be modulated does not consume the power resources of the portable electronic device.

### BRIEF DESCRIPTION OF THE DRAWINGS

The invention is now described in closer detail in connection with preferred embodiments and with reference to the accompanying drawings, in which

FIG. 1 shows a first example of an embodiment of a portable electronic device,

FIG. 2 shows a second example of an embodiment of a portable electronic device,

FIG. 3 shows an example of a structure of a signal processing chain,

FIG. 4 shows a third example of an embodiment of a portable electronic device, and

FIG. 5 shows an example of a wrist device.

### DETAILED DESCRIPTION OF THE PREFERRED EMBODIMENTS

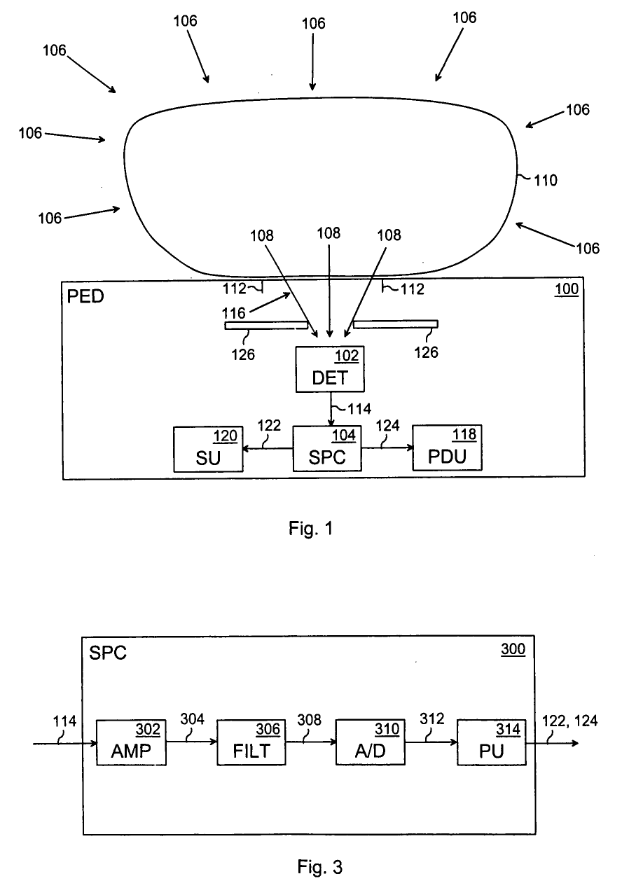

Referring to the example of FIG. 1, a portable electronic device (PED) 100 comprises an aperture 116, a radiation detector (DET) 102, and a signal processing chain (SPC) 104 connected to the radiation detector 102.

Typically, a battery or another small unit for storing electric power with a limited capacity is used as an electric power source for the portable electronic device 100.

In an embodiment, the portable electronic device 100 is a wrist device, such as a watch, wristband or a central processing unit for a performance monitor to be worn on a wrist. The central processing unit for a performance monitor may be wiredly or wirelessly connectable to an auxiliary device, such as a heart rate transmitter and/or a movement sensor.

The disclosed solution is not, however, restricted to a wrist device, but the portable electronic device 100 may be e.g. a device to be worn on a finger, or a device to be worn on the head, such as a helmet or a sweatband.

The aperture 116 allows surrounding optical radiation 108 modulated by tissue 110 of a user of the portable electronic device 100 to access the portable electronic device 100. The aperture 116 may be an opening provided in the frame of the portable electronic device 100, or it may be an optical component which is permeable to or conductive of optical radiation. FIG. 1 also shows edges 112 defining the aperture 116.

In the measurement of a blood pressure pulse, the tissue 110 of the user is arranged in the vicinity of the aperture 116. The user's tissue 110 receives surrounding optical radiation 106 at different solid angles and modulates the surrounding optical radiation 106. This results in generation of modulated surrounding optical radiation 108, which is then allowed to access the radiation detector 102 through the aperture 116.

The fact that the surrounding optical radiation 106 becomes modulated in the tissue 110 is based on variation in the distribution of blood in the tissue as a result of a change in blood pressure. The surrounding optical radiation 108 is absorbed into the blood in the tissue, so the modulation of the surrounding optical radiation 106 contains information on the blood pressure pulse.

The radiation detector 102 receives the surrounding optical radiation 108 modulated by the tissue 110 and generates an electrical signal 114. The electrical signal 114 is proportional to the intensity of the surrounding optical radiation 108 modulated by the tissue 110, and thus characterizes the blood pressure pulse.

The radiation detector 102 is e.g. an infrared detector whose operating range may be in the vicinity of 850 nm. The disclosed solution is not, however, limited to infrared detectors or the disclosed wavelength range, but any light-sensitive detector whose wavelength range overlaps with the absorption band of the tissue 110 may be used as the radiation detector 102.

The electrical signal 114 is conveyed to the signal processing chain 104 which is connected to the radiation detector 102 and which generates blood pressure pulse information 122, 124 from the electric signal 114. Such blood pressure pulse information may comprise blood pressure pulse timing, blood pressure pulse width and/or blood pressure pulse surface area.

The aperture 116, the radiation detector 102, and the signal processing chain 104 are dimensioned such that the optical measurement of a blood pressure pulse may be carried out by using an ambient radiation source, such as the sun, artificial lighting or any external radiation source with a suitable intensity and wavelength band. When the surrounding optical radiation 106 is used, the amount of optical radiation to be modulated is typically smaller than when a prior art radiation source integrated into the portable electronic device is used. Thus, when the surrounding optical radiation 106 is used, it is advisable to make the signal processing chain 104 sensitive enough so as to enable blood pressure pulse information 122, 124 to be produced from the electrical signal 114 generated by the modulated surrounding optical radiation 108.

Referring further to the example of FIG. 1, in an embodiment the portable electronic device 100 comprises an aperture stop 126 to reduce access of diffused light to the radiation detector 102. The aperture stop 126 may be integrated e.g. into a permeable optical component provided in the frame of the portable electronic device 100 or the radiation detector 102.

Referring further to the example of FIG. 1, in an embodiment the portable electronic device 100 comprises a pulse determination unit (PDU) 118 to determine the user's pulse from the blood pressure pulse information 124. The pulse determination unit 118 determines, for instance, time intervals between successive blood pressure pulses and calculates a pulse frequency from the time intervals. The pulse frequency may be displayed to the user via a user interface of the portable electronic device 100.

Referring further to the example of FIG. 1, in an embodiment the portable electronic device 100 comprises a signaling unit (SU) 120 which gives the user a signal when a blood pressure signal has been measured successfully. The signaling unit 120 may generate a voice and/or light signal, on the basis of which the user may move his or her body part being measured away from the vicinity of the portable electronic device 100.

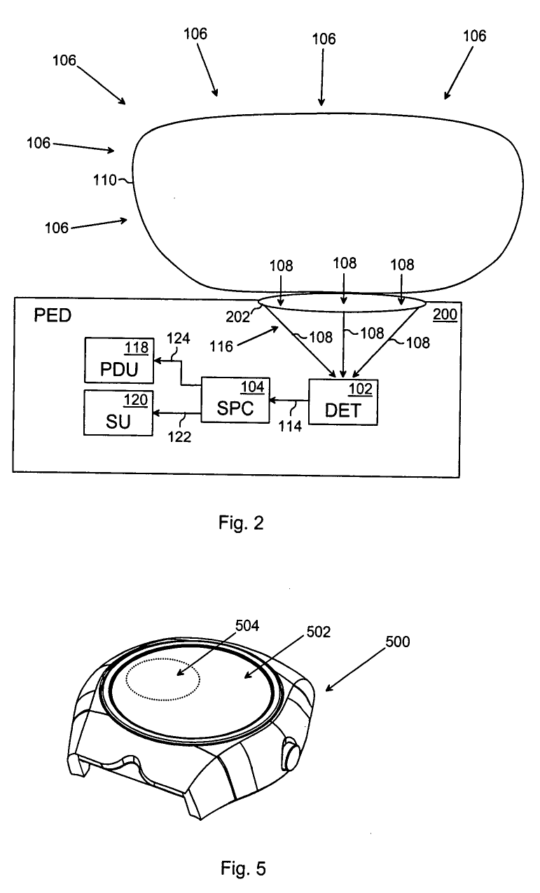

Referring to the example of FIG. 2, in an embodiment the portable electronic device 200 comprises focusing optics 202 for focusing the surrounding optical radiation 108 modulated by the user's tissue 110 on the radiation detector 102. The focusing optics 202 enables a better sensitivity to be achieved for the optical measurement. The focusing optics 202 may be integrated e.g. into a front panel of a wrist device.

The focusing optics 202 may be implemented e.g. by a ball lens whose focus resides at the light-sensitive component of the radiation detector 102.

Referring to the example of FIG. 3, a signal processing chain 300 may comprise an amplifier circuit (AMP) 302, a filtering circuit (FILT) 306, an analog-to-digital converter (A/D) 310, and a processing unit (PU) 314.

The amplifier circuit 302 receives an electrical signal 114 generated by a radiation detector 102 and amplifies a signal 304 amplified from the electrical signal 114. The amplifier circuit 302 may include an AGC amplifier (AGC, Automatic Gain Control). The amplification of the amplification circuit 302 is set according to the intensity of the modulated surrounding optical radiation 108. A typical amplification of the amplification circuit 302 may be 90 to 110 dB.

The amplified signal 304 may be conveyed to a filtering circuit 306 which attenuates desired frequency components of the amplified signal 304. The filtering circuit 306 may be e.g. a band-pass filter whose pass band is e.g. 0.3 to 6 Hz. The filtering circuit 306 supplies the filtered signal 308 to the analog-to-digital converter 310.

The analog-to-digital converter 310 converts the filtered signal 308 into a digital signal 312. The dynamic operating range of the analog-to-digital converter 310 is selected to correspond with the dynamic range of the modulated surrounding optical radiation 108.

The analog-to-digital converter 310 supplies the digital signal 312 to the processing unit 314 which subjects the digital signal 312 to a computer process. The computer process may comprise digital filtering, pulse identification as well as determination of pulse characteristics, such as pulse timing and/or pulse width.

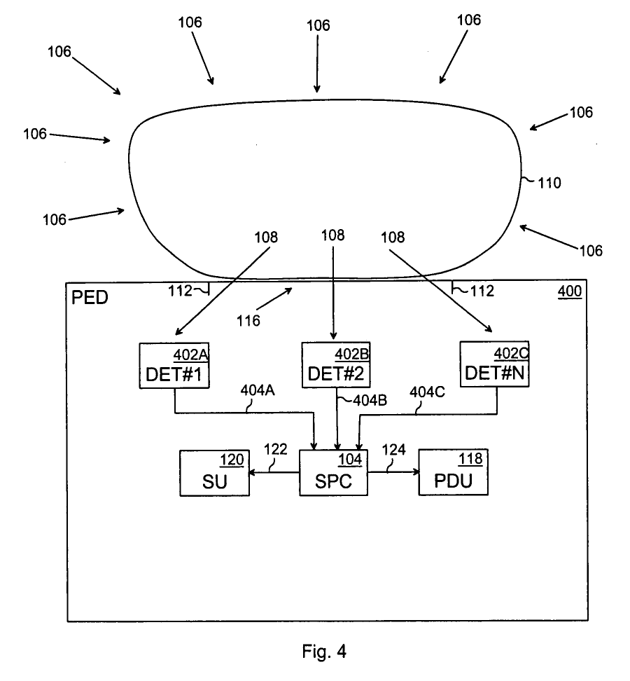

Referring to FIG. 4, in one embodiment a portable electronic device 400 comprises a plurality of radiation detectors 402A to 402C connected to a signal processing chain 102, each of which radiation detectors generating an electrical signal 404A to 404C from the surrounding optical radiation 108 modulated by the user's tissue 110. The signal processing chain 104 may select radiation detectors 402A to 402C to be used in the optical measurement on the basis of the electrical signals 404A to 404C. The signal processing chain 104 may comprise e.g. a comparing element which compares the intensity of the electrical signals 404A to 404C and selects e.g. the radiation detectors 402A to 402C which produce the most intensive electrical signal or signals 404A to 404C as detectors to be used in the measurement.

Referring to FIG. 5, a wrist device 500 comprises a front panel 502 comprising an optically permeable segment 504. The optically permeable segment 504 may operate as an aperture 116 according to FIG. 1, in which case a user may place his or her finger on to the optically permeable segment 504. Then, the surrounding optical radiation 106 hits the finger's tissue, which modulates the surrounding optical radiation 108 directed at the radiation detector 102 residing underneath the front panel 502 of the wrist device 500. The optically permeable segment 504 may be provided with visual marks or embossings to guide the user in finding the correct place for his or her finger on the front panel 502.

Although the invention has been described above with reference to the example according to the accompanying drawings, it is clear that the invention is not restricted thereto but can be modified in many ways within the scope of the attached claims.

What is claimed is:

1. A portable electronic device for optically measuring a blood pressure pulse from a user's tissue, wherein the portable electronic device comprises:
an aperture for allowing surrounding optical radiation modulated by the user's tissue to access the portable electronic device;
a radiation detector for generating an electrical signal from the surrounding optical radiation modulated by the user's tissue; and
a signal processing chain connected to the radiation detector and configured to generate blood pressure pulse information from the electrical signal.

2. A portable electronic device as claimed in claim 1, wherein the portable electronic device further comprises stopper means for reducing access of diffused light to the radiation detector.

3. A portable electronic device as claimed in claim 1, wherein the portable electronic device further comprises focusing means for focusing the surrounding optical radiation modulated by the user's tissue on the radiation detector.

4. A portable electronic device as claimed in claim 1, wherein the portable electronic device further comprises signaling means configured to give the user a signal when a blood pressure pulse has been measured successfully.

5. A portable electronic device as claimed in claim 1, wherein the portable electronic device further comprises pulse determination means, connected to the signal processing chain, for determining the user's pulse from the blood pressure pulse information.

6. A portable electronic device as claimed in claim 1, wherein the portable electronic device is a wrist device.

7. A portable electronic device as claimed in claim 6, wherein the wrist device comprises a front panel arranged to receive the user's finger whose tissue modulates the surrounding optical radiation.

8. A portable electronic device as claimed in claim 1, wherein the portable electronic device comprises a plurality of radiation detectors connected to the signal processing chain, each radiation detector being configured to generate an electrical signal from the surrounding optical radiation modulated by the user's tissue and the signal processing chain being configured to select radiation detectors to be used for the optical measurement on the basis of the electrical signals.
